(19) United States
(12) Patent Application Publication
(10) Pub. No.: US 2015/0090271 A1
(43) Pub. Date: Apr. 2, 2015

(54) IMPACT FORCE DAMPENING OF SPRING RELEASE

(71) Applicants: Julian Cruzada, San Jose, CA (US); Christopher A. Stout, San Bruno, CA (US); Stephan L. Wartinger, Santa Clara, CA (US)

(72) Inventors: Julian Cruzada, San Jose, CA (US); Christopher A. Stout, San Bruno, CA (US); Stephan L. Wartinger, Santa Clara, CA (US)

(21) Appl. No.: 14/043,695

(22) Filed: Oct. 1, 2013

(51) Int. Cl. A61F 6/00

(52) U.S. Cl. CPC A61F 6/005
USPC 128/831

(57) ABSTRACT

A delivery system and spring assembly are disclosed. In an embodiment, the delivery system includes a spring-loaded release mechanism movable from a first position to a second position to cause withdrawal of a release wire form an occlusion device. In an embodiment, a spring assembly includes a damper ring that rotates along a helical track of a bore upon linear contraction of an extension spring in order to dampen the spring force of the extension spring.

### BACKGROUND

Female contraception and sterilization may be enabled by transcervically introduced fallopian tube inserts. Devices, systems and methods for contraceptive approaches have been described in various patents and patent applications assigned to the present assignee. For example, U.S. Pat. No. 6,526,979, U.S. Pat. No. 6,634,361, U.S. Pat. No. 8,360,064, and U.S. Pat. No. 8,434,489 describe transcervically introducing an occlusion device (also referred to as implant and insert) through an ostium of a fallopian tube and mechanically anchoring the occlusion device within the fallopian tube. Tissue in-growth into the occlusion device induces long-term contraception and/or permanent sterilization.

A delivery system for inserting an occlusion device is described in U.S. Pat. No. 8,360,064 in which a thumbwheel release mechanism is rotated by a user to cause an expandable portion of the occlusion device to expand and a core wire to withdraw from the expanded occlusion device to disengage the expanded occlusion device from the delivery catheter. In order to accurately deploy the occlusion device from the delivery system, the user maintains the delivery system at the target location while rotating the thumbwheel.

### SUMMARY

Delivery systems, spring assemblies, and methods of use to facilitate occlusion of a body lumen, such as a fallopian tube, are disclosed. In an embodiment, a delivery system includes a delivery catheter, an occlusion device, and a handle coupled with the delivery catheter. The handle includes a spring-loaded release mechanism that is movable from a first position to a second position to cause withdrawal of a release wire from the occlusion device. The occlusion device may include a self-expandable member. A half band can be coupled with the self-expandable member, and the release wire threaded through the half band to hold the self-expandable member in a wound-down static state prior to movement of the spring-loaded release mechanism from the first position. Upon movement of the spring-loaded release mechanism from the first position to the second position the release wire is withdrawn from the half-band causing the self-expandable member to expand. In an embodiment, movement of the spring-loaded release mechanism from the first position to the second position causes a distal end of the release wire to be withdrawn into a distal end of the delivery catheter. The delivery system may additionally include a first release mechanism, where movement of the first release mechanism from a first position to a second position withdraws a rack that is coupled with the delivery catheter to withdraw the delivery catheter from over the occlusion device.

The release wire may be coupled with a delivery wire holder that is coupled to a spring assembly. In an embodiment, the spring-loaded release mechanism is a push button, and movement of the push button form the first position to the second position allows the spring assembly to withdraw the delivery wire holder and cause withdrawal of the release wire from the occlusion device. A core wire may additionally be coupled with the delivery wire holder. In an embodiment, movement of the spring-loaded release mechanism from the first position to the second position allows the spring assembly to withdraw the delivery wire holder and cause simultaneous withdrawal of the release wire and the core wire from the occlusion device.

In an embodiment, the spring assembly includes a damper ring and an extension spring. The spring assembly may further include a bore, a helical track along a wall of the bore, a piston, and an extension spring. Linear contraction of the extension spring causes axial motion of the piston and the damper ring within the bore and rotational motion of the damper ring along the helical track. In this manner, a spring force associated with linear contraction of the extension spring is dampened by a drag force associated with rotational motion of the damper ring along a helical track in a bore. This piston may be the delivery wire holder of the delivery system. Alternatively, the spring assembly can be a self-contained modular spring assembly that includes a coupler coupled with piston and extending outside of a housing. In this manner, the self-contained modular spring assembly can be a separate component, for example, that could be inserted into the delivery system.

The bore and helical track can be formed in a variety of configurations. For example, the bore and helical track can be formed from a casing of the handle. In an embodiment, the damper ring includes one or more lugs that travel within a matching quantity of paths in the helical track. The damper ring or bore can be designed to an increasing drag force coefficient as the damper ring travels axially within the bore. In an embodiment, a pitch of the helical track is tightened along an axial length of the bore. In an embodiment, an inside diameter of the bore is reduced along an axial length of the bore. In an embodiment, the damper ring includes slots around a circumference of a tubular body of the damper ring. In an embodiment, the pitch of the helical track is tightened along an axial length of the bore, an inside diameter of the bore is reduced along the axial length of the bore, and the damper ring includes slots around a circumference of the tubular body of the damper ring. A number of additional dampening mechanisms and combinations are possible. For example, a surface roughness of the helical track can be increased along an axial length of the bore, or a dampening grease can be located within the helical track where a viscosity of the dampening grease increases along an axial length of the bore. In an embodiment, an elastic band couples the delivery wire holder to the handle to provide an increasing resistance (drag) force coefficient as the extension spring linearly contracts.

### BRIEF DESCRIPTION OF THE DRAWINGS

FIG. 1 is an illustration of a delivery system in accordance with an embodiment of the invention.

FIG. 2 is a cross-sectional side view illustration of an inner catheter and handle of a delivery system in accordance with an embodiment of the invention.

FIG. 3 is a side view illustration of a delivery catheter and rack of a delivery system in accordance with an embodiment of the invention.

FIG. 4 is a cross-sectional side view illustration of an inner catheter and delivery catheter of a delivery system in a first position in accordance with an embodiment of the invention.

FIG. 5 is a cross-sectional side view illustration of an inner catheter and delivery catheter of a delivery system in a second position in accordance with an embodiment of the invention.

FIG. 6A is a cross-sectional side view illustration of a spring-loaded release mechanism, release wire, and core wire of a delivery system in a first position in accordance with an embodiment of the invention.

FIG. 6B is a close-up view of a delivery wire holder coupled with a spring assembly of the delivery system illustrated in FIG. 6A, in accordance with an embodiment of the invention.

FIG. 7A is a cross-sectional side view illustration of a spring-loaded release mechanism, release wire, and core wire of a delivery system in a second position in accordance with an embodiment of the invention.

FIG. 7B is a close-up view of a spring assembly of the delivery system illustrated in FIG. 7A, in accordance with an embodiment of the invention.

FIG. 8A is a side view illustration of an occlusion device and distal portion of a delivery system prior to withdrawal of a delivery catheter in accordance with an embodiment of the invention.

FIG. 8B is a side view illustration comparing the relative dimensions of an expanded occlusion device side-by-side with the distal portion of a delivery system prior to withdrawal of a delivery catheter in accordance with an embodiment of the invention.

FIG. 9A is a perspective view illustration of a wound-down self-expandable member and release wire threaded through a half band in accordance with an embodiment of the invention.

FIG. 9B is a cross-sectional view of the illustration of FIG. 9A in accordance with an embodiment of the invention.

FIG. 10A is a side view illustration of an occlusion device and distal portion of a delivery system after withdrawal of a delivery catheter in accordance with an embodiment of the invention.

FIG. 10B is a side view illustration comparing the relative dimensions of an expanded occlusion device side-by-side with the distal portion of a delivery system after withdrawal of a delivery catheter in accordance with an embodiment of the invention.

FIGS. 11A-11B are cross-sectional side view illustrations of withdrawing a friction fit core wire from an inner coil in accordance with an embodiment of the invention.

FIG. 11C is a side view illustration comparing the relative dimensions of a disengaged and expanded occlusion device side-by-side with the distal portion of a delivery system after withdrawal of the release wire and core wire in accordance with an embodiment of the invention.

FIG. 12 is a schematic side view illustration of spring assembly including an extension spring, damper ring, and helical track in accordance with an embodiment of the invention.

FIG. 13A is a side view illustration of a helical track formed in a sidewall of a bore in accordance with an embodiment of the invention.

FIG. 13B is a cross-sectional side view illustration of a helical track formed in a sidewall of a bore in accordance with an embodiment of the invention.

FIG. 14A is a top view illustration of a damper ring including one or more lugs in accordance with an embodiment of the invention.

FIG. 14B is a side view illustration of a damper ring including one or more lugs in accordance with an embodiment of the invention.

FIG. 14C is a close-up view of a lug within a helical track in accordance with an embodiment of the invention.

FIGS. 15A-15C are schematic side view illustrations of a delivery wire holder and damper ring traveling along an axial length of a bore during linear contraction of an extension spring while the damper ring rotates about a double helical track in the bore in accordance with an embodiment of the invention.

FIGS. 16A-16B are side view illustrations of a helical track with differing pitch angles in accordance with embodiments of the invention.

FIG. 17 is a side view illustration of a reduced diameter along an axial length of a bore in accordance with an embodiment of the invention.

FIG. 18 is an isometric view illustration of a damper ring including slots around a circumference of a tubular body of the damper ring in accordance with an embodiment of the invention.

FIG. 19 is a side view illustration of a spring assembly including an elastic band that couples a delivery wire holder to a handle in accordance with an embodiment of the invention.

FIG. 20A is a close-up view of a dampening grease within a helical track in accordance with an embodiment of the invention.

FIG. 20B is a side view illustration of a helical grease within a helical track in which a viscosity of the dampening grease increases along an axial length of the bore in accordance with an embodiment of the invention.

FIG. 21A is a close-up view of textured surfaces of a cylindrical bore, helical track, and mating surfaces of a damper ring in accordance with an embodiment of the invention.

FIG. 21B is a side view illustration of increased texture roughness along an axial length of the bore in accordance with an embodiment of the invention.

FIG. 22A is a perspective view of a self-contained modular spring assembly in accordance with an embodiment of the invention.

FIG. 22B is a schematic side view illustration of a self-contained modular spring assembly in accordance with an embodiment of the invention.

### DETAILED DESCRIPTION

Embodiments of the present invention generally provide a delivery system for delivery of an occlusion device to a body lumen. More specifically, some embodiments provide a spring-loaded release mechanism within a handle of the delivery system. In accordance with some embodiments, various dampening configurations can be incorporated in a spring assembly to dampen an impact of a spring force used for delivery of the occlusion device.

Various embodiments and aspects will be described with reference to details discussed below and the accompanying drawings will illustrate the various embodiments. The following description and drawings are illustrative of the invention and are not to be construed as limiting the invention. Numerous specific details are described to provide a thorough understanding of various embodiments of the present invention. However, in certain instances, well-known or conventional details are not described in order to provide a concise discussion of embodiments of the present invention.

In an embodiment a delivery system includes a delivery catheter, an occlusion device, and a handle coupled with the delivery catheter. The handle includes a spring-loaded release mechanism movable from a first position to a second position to cause withdrawal of a release wire from the occlusion device. The spring-loaded release mechanism may be a push button in some embodiments, and movement of the push button form the first position to the second position may cause a spring assembly to withdraw a delivery wire holder and cause withdrawal of a release wire from the occlusion device. Movement of the spring-loaded release mechanism, e.g. push button, from the first position to the second position may additionally cause simultaneous withdrawal of a core wire from the occlusion device. A spring assembly can be incorporated into the handle or alternatively as a self-contained modular unit that can be placed into the handle. In accordance with embodiments of the invention, the spring assembly includes a damper ring connected with a piston or delivery wire holder and an extension spring coupled with the piston or delivery wire holder. Linear contraction of the extension spring causes axial motion of the piston or delivery wire holder and the damper ring within a bore, and rotational motion of the damper ring along a helical track. The rotational motion of the damper ring along the helical track can function to provide a dampening force on the spring force associated with the extension spring.

In one aspect, embodiments of the invention describe a delivery system and spring assembly that can quickly withdraw components such as a delivery wire and core wire from an occlusion device to enable rapid deployment. A handle of the delivery system may include a spring-loaded release mechanism, such as a push button, that can be quickly and easily activated by a user upon tracking the occlusion device to the target location, such as within a fallopian tube. In an embodiment, actuation of the button releases a spring assembly, allowing linear contraction of an extension spring to cause rapid withdrawal of the delivery wire and core wire from the occlusion device, resulting in rapid and accurate deployment of the occlusion device from the delivery system.

In another aspect, embodiments of the invention describe a delivery system and spring assembly that dampens a spring force associated with linear contraction of an extension spring. In accordance with embodiments of the invention, a certain spring force is required from extension spring to overcome the forces retaining the delivery wire and core wire within the occlusion device. It is possible that generation of such a spring force, if not dampened, could result in an impact force on the handle that could potentially be felt by the user, potentially causing the user to move the delivery system during delivery resulting in inaccurate deployment location of the occlusion device. Additionally, it is possible such an impact force could also be transferred to the occlusion device resulting in launching the occlusion device distally away from the delivery system. In accordance with embodiments of the invention, the spring assembly dampens the spring force associated with linear contraction of the extension spring. For example, dampening may be in the form of a drag force associated with rotational motion of a damper ring along a helical track in a bore. In some embodiments, the spring assembly provides a gradually increased dampening as the extension spring linearly contracts to gradually slow down motion of the contracting extension spring, thereby reducing the impact force. Additional dampening forces that may be included in the alternative or in any combination, include variable pitch of the helical track, reduced inside diameter of the bore along an axial length of the bore, a slotted damper ring, controlled surface roughness of the helical track along an axial length of the bore, controlled viscosity of a dampening grease along an axial length of the bore, a fluid within the bore, and an elastic band coupled to the piston or release wire holder to provide a resistance to the linear contraction of the extension spring. In one exemplary embodiment the spring assembly includes a combination of a helical track, reduced inside diameter of the bore along an axial length of the bore, and a slotted damper ring, though other combinations are possible.

In some embodiments, the spring assembly provides an increased drag force coefficient in the direction of the linearly contracting extension spring. In accordance with embodiments of the invention, it is understood that a drag force may be a responsive force to the spring force, which may decrease as an extension spring linearly contracts. Accordingly, the term drag force coefficient as used herein does not necessarily correspond to the drag force applied, in particular where the spring force decreases as an extension spring linearly contracts. However, in embodiments where the spring assembly provides an increased drag force coefficient, an amount of drag force may increase if a constant spring force were supplied over the linear contraction distance of the extension spring. In an embodiment, the extension spring can be replaced with another contraction mechanism.

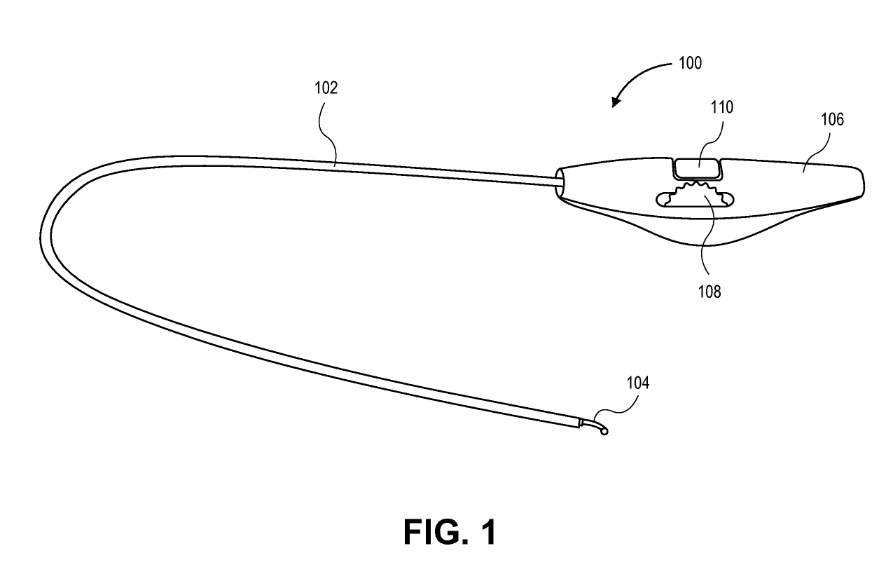

FIG. 1 is an illustration of a delivery system in accordance with an embodiment of the invention. As illustrated, delivery system 100 can include a delivery catheter 102, an occlusion device 104, and a handle 106 coupled with the delivery catheter 102. The handle 106 may include a first release mechanism 108 and a second release mechanism 110. In the particular embodiment illustrated, the first release mechanism 108 is a thumbwheel and the second release mechanism 110 is a push button. In accordance with embodiments of the invention, movement of the first release mechanism 108 from a first position to a second position may cause withdrawal of the delivery catheter 102 to expose an unexpanded occlusion device 104. For example, movement of the first release mechanism may include rolling back a thumbwheel, as illustrated. After actuation of the first release mechanism 108 the second release mechanism 110 may be moved from a first position to a second position to cause withdrawal of a release wire from the occlusion device 104 allowing the occlusion device 104 to expand. For example, movement of the second release mechanism 110 may include depressing a push button, as illustrated. Actuation of the second release mechanism 110 may additionally simultaneously withdraw the release wire and a core wire from the occlusion device, where withdrawal of the core wire deploys the occlusion device 104 from the delivery system 100.

More detailed description and illustrations of the individual components of delivery system 100 are provided in the following description and figures. In interest of clarity specific components may have been removed from some of the illustrations in order to more clearly point out the operability of certain components and interoperability of respective components within the delivery system. Accordingly, the following description and drawings are illustrative of the invention and are not to be construed as limiting the invention.

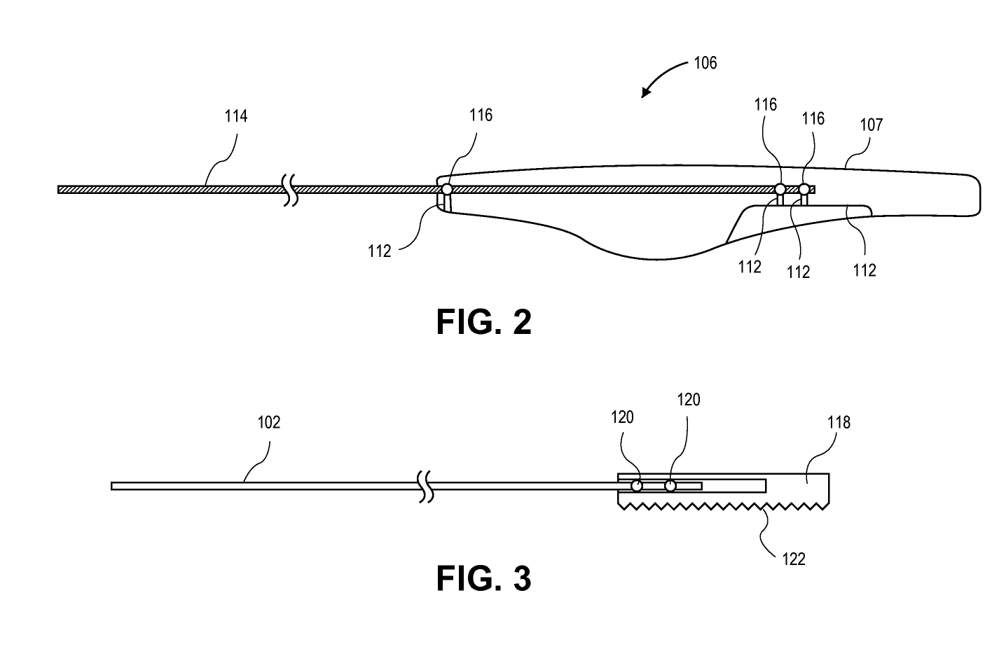

FIG. 2 is a cross-sectional side view illustration of an inner catheter and handle of a delivery system in accordance with an embodiment of the invention. In particular, the cross-sectional side view illustration provided in FIG. 2 shows a cross-section of the casing 107 of the handle 106 in which several features 112 are formed. Features 112 may be protrusions from a sidewall of the casing 107. For example, casing 107 including protruding features 112 can be a single molded piece of material. The inner catheter 114 may be bonded to the casing 107 of handle 106 at one or more locations using an adhesive bonding material 116, and extends distally from the handle 106. In this manner, the position of the inner catheter 114 relative to the handle 106 can be permanently fixed.

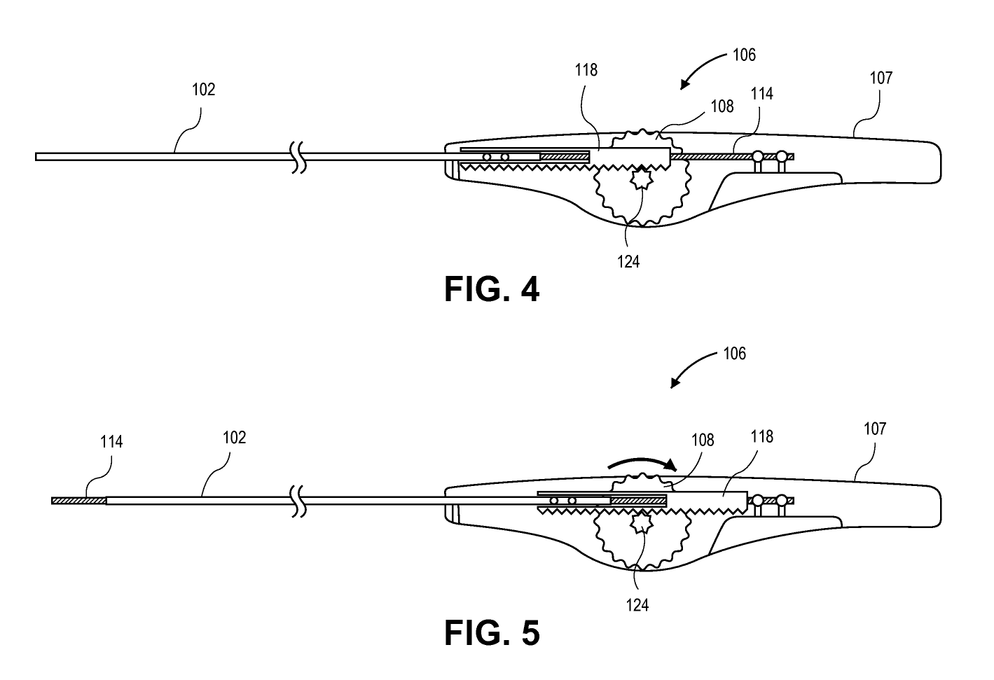

Referring now to FIGS. 3-5 the relative position of the inner catheter 114 and delivery catheter 102 are described and illustrated upon actuation of the first release mechanism 108, in accordance with embodiments of the invention. FIG. 3 is a side view illustration of a delivery catheter 102 and rack 118 of a delivery system in accordance with an embodiment of the invention. As shown the delivery catheter 102 may be bonded to the rack 118 using an adhesive bonding material 120 so that the relative position of the delivery catheter 102 is permanently fixed relative to the rack 118. The rack may include a plurality of teeth 122 that are operatively coupled with a pinion 124 as described with regard to FIGS. 4-5.

FIG. 4 is a cross-sectional side view illustration of an inner catheter and delivery catheter 102 of a delivery system 100 in a first position in accordance with an embodiment of the invention. FIG. 5 is a cross-sectional side view illustration of an inner catheter 114 and delivery catheter 102 of a delivery system 100 in a second position in accordance with an embodiment of the invention. As illustrated in FIGS. 4-5 the first release mechanism 108, illustrated as a thumbwheel, can be actuated between a first and second position to withdraw the delivery catheter 102 from over the inner catheter 114 so that the inner catheter 114 extends distally from a distal end of the delivery catheter 102. In an embodiment, the first release mechanism 108 includes a pinion 124. Upon actuation of the first release mechanism, e.g. rolling back the thumbwheel, the pinion 124 is rotated and operably engages the teeth 122 of the rack 118 to draw the rack proximally into the casing 107 of handle 106. Consequently, the delivery catheter 102 is also withdrawn proximally in the casing 107 of handle 106, while the inner catheter 114 remains in a fixed position relative to the handle.

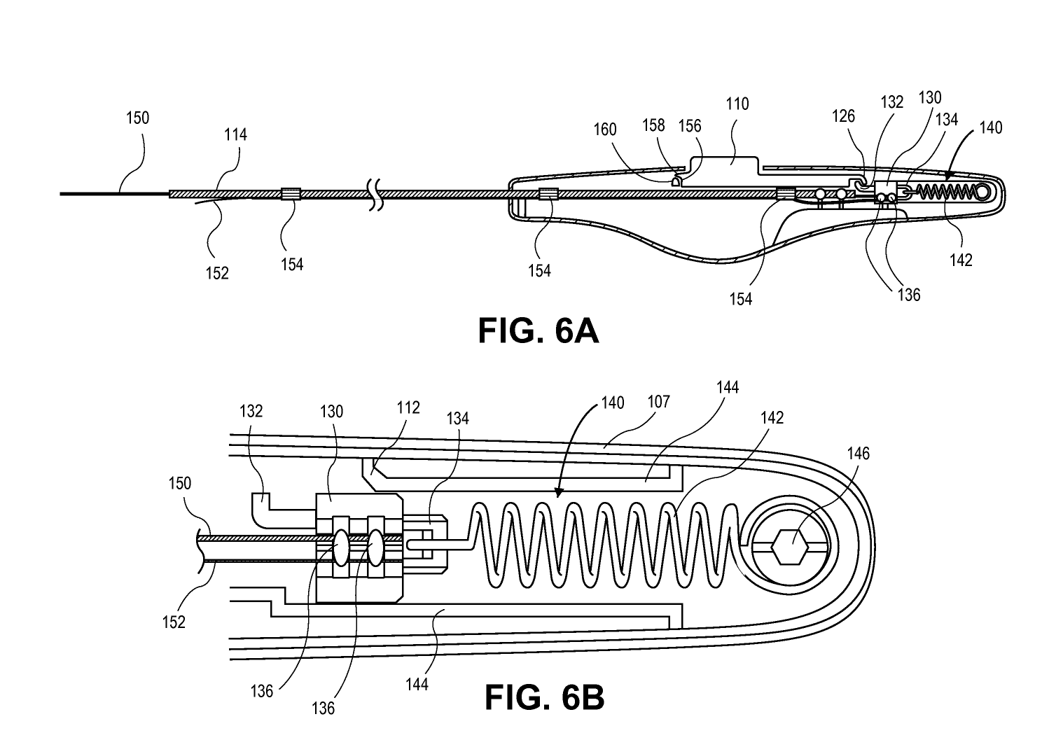

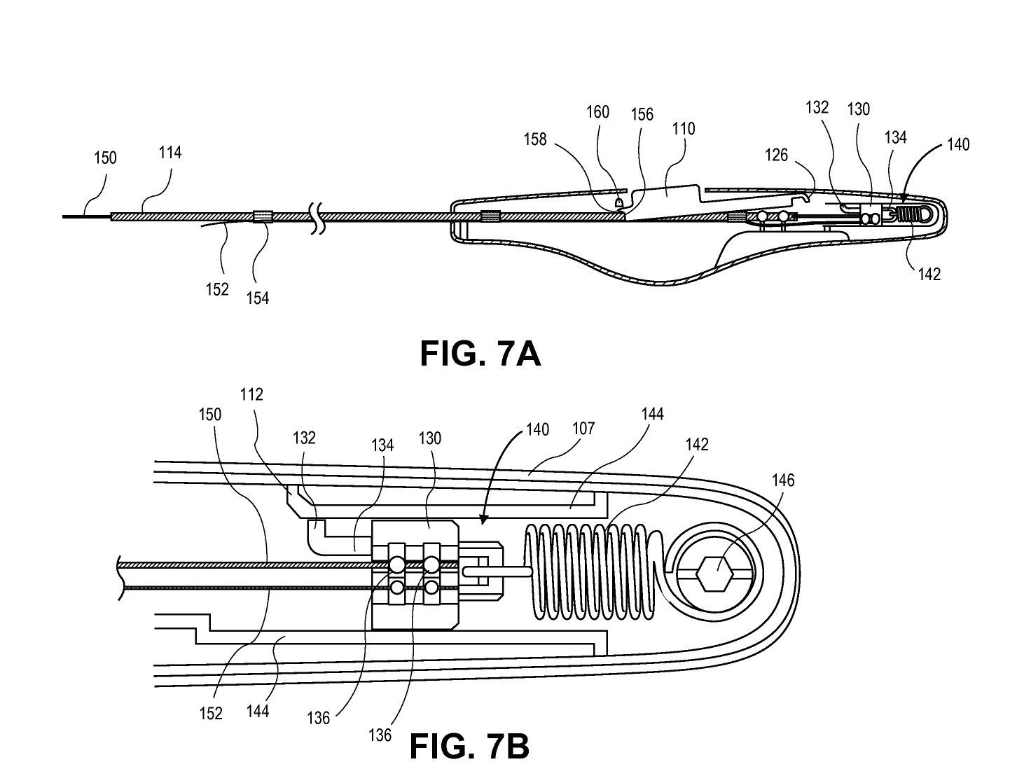

Referring now to FIGS. 6A-7B the relative position of the inner catheter, release wire, and core wire are described and illustrated upon actuation of the second release mechanism 110, in accordance with embodiments of the invention. FIG. 6A is a cross-sectional side view illustration of a spring-loaded release mechanism, release wire, and core wire of a delivery system in a first position in accordance with an embodiment of the invention. As illustrated, the second release mechanism 110 is in the form of a push button. In an embodiment, the release wire 152 and core wire 150 are connected to a delivery wire holder 130 located within the handle. A suitable adhesive material 136 may be used to bond the release wire 152 and core wire 150 to the delivery wire holder 130. In the particular embodiment illustrated, the core wire 150 extends through the inner catheter 114 and extends distally beyond the inner catheter 114. In an embodiment, the release wire 152 runs outside of inner catheter 114. For example, the release wire 152 can be secured along the inner catheter 114 using a series of restraining collars 154, such as heat shrink tubing or another suitable mechanism so that the release wire 152 can be withdrawn proximally within the handle while the inner catheter 114 remains in a fixed position. As illustrated, the release wire 152 does not extend distally past a distal end of the inner catheter 114 when the second release mechanism 110 is in the first position.

In accordance with embodiments of the invention, the second release mechanism 110 may be spring-loaded, so that upon movement of the second release mechanism, e.g. depressing the push button, the release wire 152 is withdrawn from the occlusion device. In an embodiment, a core wire 150 may be simultaneously withdrawn from the occlusion device with the release wire 152. As shown in FIG. 6A, a latch 132 connected with the delivery wire holder 130 is operable coupled with a latch 126 of the second release mechanism 110.

FIG. 6B is a close-up view of a delivery wire holder 130 coupled with a spring assembly 140 of the delivery system illustrated in FIG. 6A, in accordance with an embodiment of the invention. In an embodiment, the spring assembly 140 includes an extension spring 142 and a bore 144 through which the delivery wire holder 130 can be retracted. In the particular embodiment illustrated in FIG. 6B, the extension spring 142 is in an extended configuration and connected to an eyelet or hook 134 at a distal end, and a hub 146 at a proximal end of the extension spring. As previously described, a number of protruding features 112 can be formed in a sidewall of the casing 107 of the handle. In an embodiment, the bore 144 is a protruding feature from the casing 107. In an embodiment, the hub 146 is a protruding feature from the casing 107. It is to be appreciated however, that the bore 144 and hub can be formed separately from the casing 107 in other embodiments.

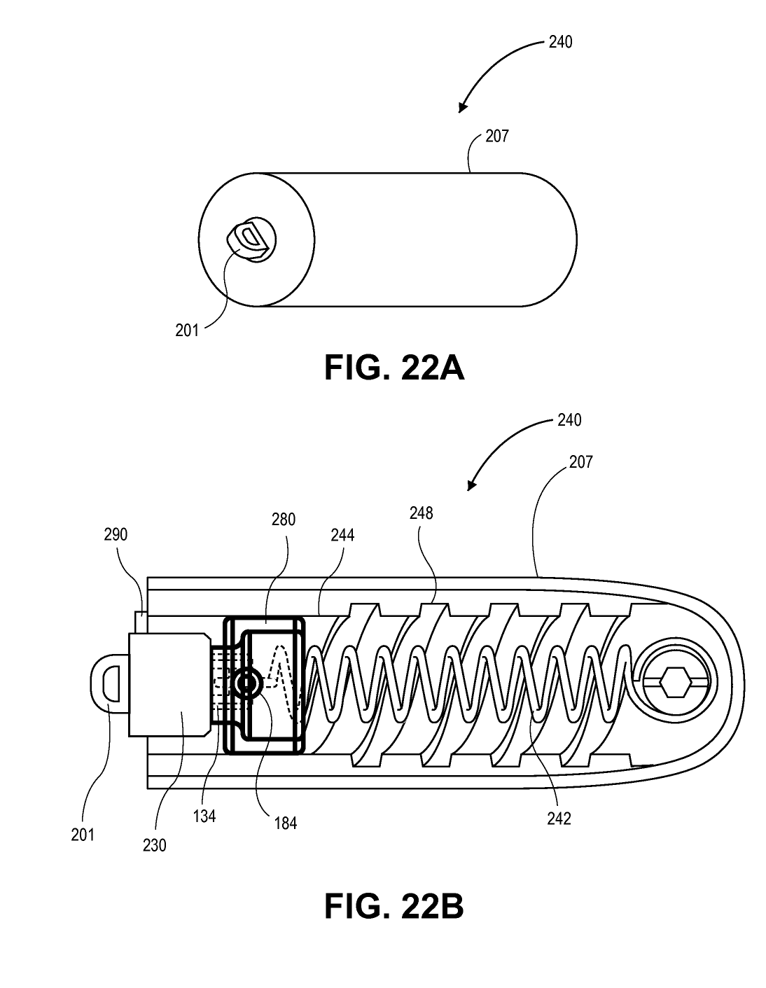

In yet another embodiment, a self-contained modular spring assembly 240 such as the one illustrated in FIG. 22B and described in further detail below can be secured within the casing 107 of handle 106 and operably coupled with the delivery wire holder 130 to function similarly as the spring assembly 140.

Referring now to FIG. 7A a cross-sectional side view illustration is provided of a spring-loaded release mechanism, release wire, and core wire of a delivery system in a second position in accordance with an embodiment of the invention. FIG. 7B is a close-up view of a spring assembly of the delivery system illustrated in FIG. 7A, in accordance with an embodiment of the invention. In the particular embodiment illustrated, the push button 110 (spring-loaded release mechanism) has been depressed so that a curved edge 156 of a tip 158 slides beneath a stop pin 160 to that the tip 158 becomes trapped underneath the stop pin 160 and maintain the push button 110 in the second position. Movement to the second position decouples the latches 126, 132 allowing the extended extension spring 142 to contract, drawing the delivery wire holder 130 proximally into the handle, along with the release wire 152 and core wire 150. In the particular embodiment illustrated the core wire 150 is not completely withdrawn into the inner catheter 114. However, in other embodiments the core wire may be completely withdrawn through the distal end of the inner catheter 114.

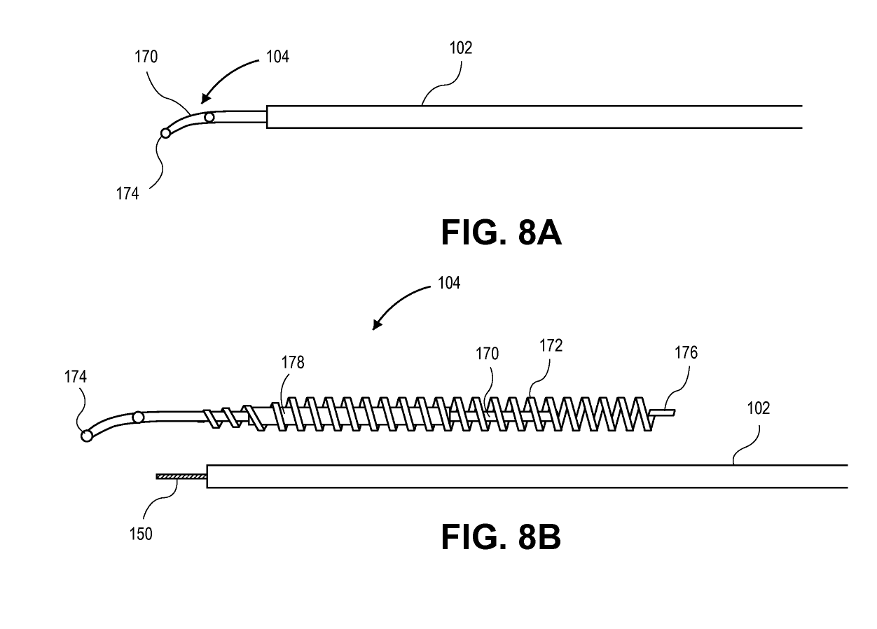

Up until this point the preceding discussion has been made without regard to the interoperability of the occlusion device with the other components of the delivery system in order to more clearly describe and illustrate the locational relationships of the delivery catheter 102, inner catheter 114, release wire 152, core wire 150, etc. The following description made with regard to FIGS. 8A-11C is made with specific reference to the occlusion device 104. In interest of clarity specific components have been removed from the illustrations in order to more clearly point out the operability of certain components and interoperability of respective components within the delivery system. Accordingly, the following description and drawings are illustrative of the invention and are not to be construed as limiting the invention.

FIG. 8A is a side view illustration of an occlusion device 104 and distal portion of a delivery system prior to withdrawal of a delivery catheter 102 in accordance with an embodiment of the invention. As illustrated, a distal portion of the occlusion device 104 may extend distally past the delivery catheter 102 to aid in tracking the delivery system 100 to a target location such as a fallopian tube. In an embodiment, the distal portion of the occlusion device includes an inner coil 170 and an atraumatic distal ball 174 secured to the inner coil 170. Referring now to FIG. 8B a side view illustration comparing the relative dimensions of an expanded occlusion device side-by-side with the distal portion of a delivery system prior to withdrawal of the delivery catheter is shown in accordance with an embodiment of the invention. In an embodiment, the occlusion device 104 includes a self-expandable member 172 and a half band 176 at a proximal end of the self-expandable member 172. In an embodiment, the self-expandable member 172 is an outer coil that surrounds an inner coil 170. A tissue ingrowth promoting agent 178 such as polyethylene terephthalate (PET) fibers can be located between the inner and outer coils to promote tissue ingrowth and permanent occlusion of the body lumen. It is to be understood that the occlusion device 104 is not actually in the expanded state prior to withdrawal of the delivery catheter. Instead, the occlusion device 104 is in the configuration illustrated and described with regard to FIG. 8A. Rather, FIG. 8B is provided to illustrate the dimensional relationship of the components within the delivery system 100. For example, in the embodiment illustrated, the distal end of the core wire 150 extends distally past the distal end of the delivery catheter 102 and inside the inner coil 170. In the embodiment illustrated, the release wire and inner catheter do not extend distally past the distal end of the delivery catheter 102.

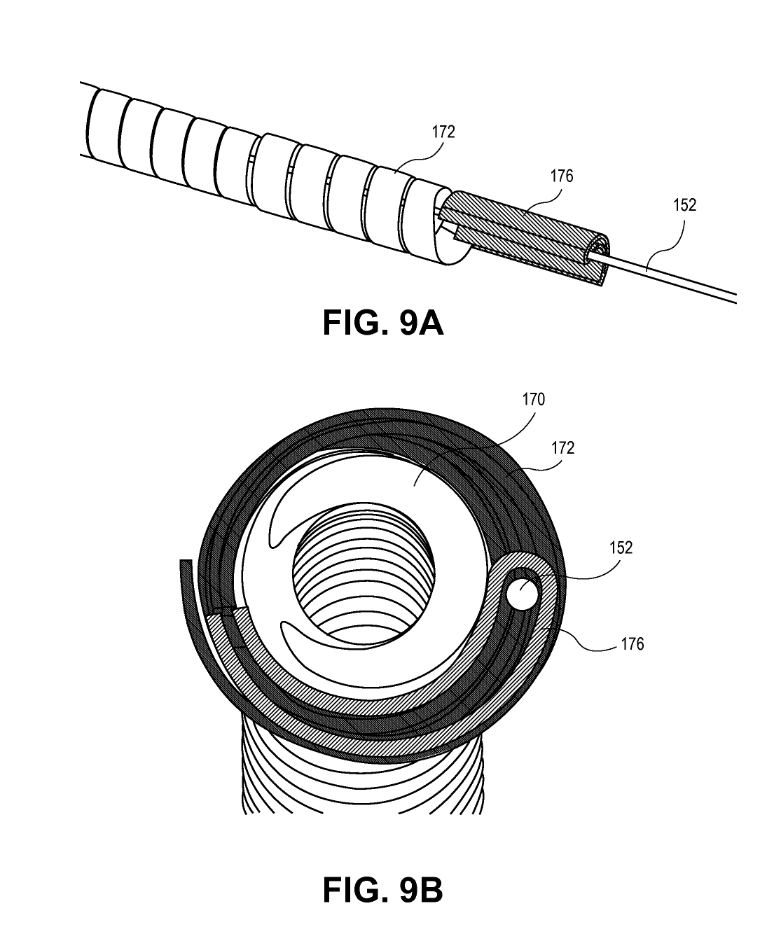

Referring now to FIGS. 9A-9B, FIG. 9A is a perspective view illustration of a wound-down self-expandable member 172 and release wire 152 threaded through a half band 176 in accordance with an embodiment of the invention; FIG. 9B is a cross-sectional view of the illustration of FIG. 9A in accordance with an embodiment of the invention. As shown the release wire 152 is threaded through the half band 176 and the self-expandable member 172 is wound down tightly. The release wire 152 holds the wound-down self-expandable member 172 in a static state so that it does not unravel. In an embodiment, the release wire 152 is also tucked underneath a portion of the self-expandable member 172 (e.g. some coils where the self-expandable member 172 is an outer coil) so that the self-expandable member 172 in turn holds the release wire 152 so that the release wire does not inadvertently come out of the half band 176. In an embodiment, the self-expandable member 172 is an outer coil, and the outer coil is would down onto an inner coil 170 with a distal portion of the release wire 152 being wrapped between the outer coil and inner coil 170 in the static state.

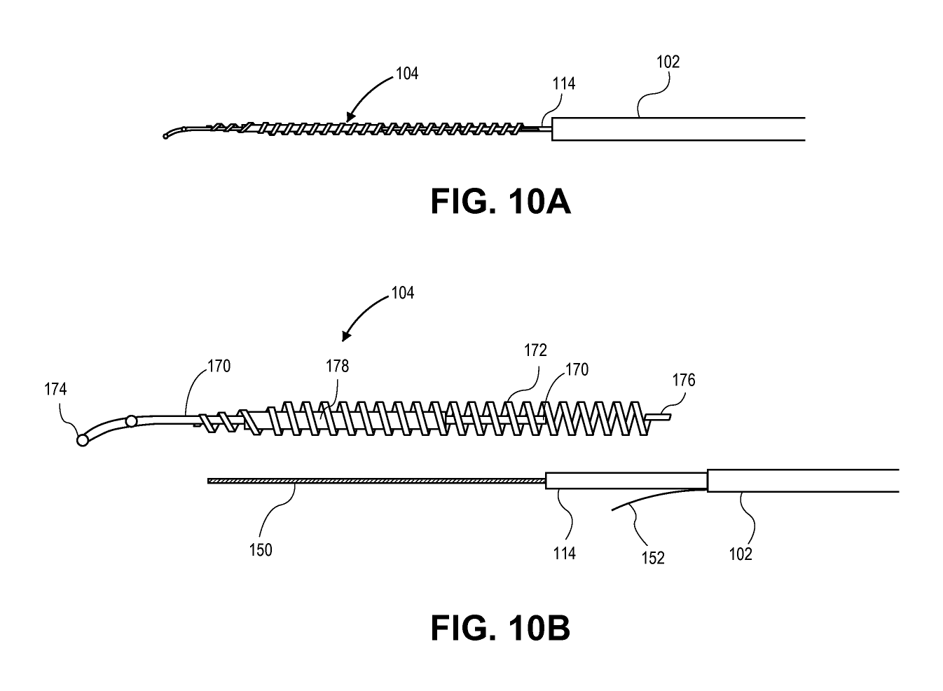

Referring now to FIG. 10A a side view illustration is shown of an occlusion device 104 and distal portion of a delivery system after withdrawal of a delivery catheter 102 in accordance with an embodiment of the invention. The particular embodiment illustrated in FIG. 10A corresponds to movement of the first release mechanism 108 (e.g. thumbwheel) from the first position to the second position, in accordance with an embodiment of the invention. As illustrated, the occlusion device 104 is completely exposed in its wound-down static state. FIG. 10B is a side view illustration comparing the relative dimensions of an expanded occlusion device side-by-side with the distal portion of a delivery system after withdrawal of a delivery catheter in accordance with an embodiment of the invention. It is to be understood that the occlusion device 104 is not actually in the expanded state upon withdrawal of the delivery catheter. Instead, the occlusion device 104 is in the wound-down configuration illustrated and described with regard to FIG. 10A. Rather, FIG. 10B is provided to illustrate the dimensional relationship of the components within the delivery system 100. For example, in the embodiment illustrated, the distal end of the core wire 150 extends through the inner coil 170 to a distal portion of the inner coil 170 similarly as described with regard to FIG. 8B. Furthermore, in the embodiment illustrated, the distal end of the inner catheter 114 abuts against a proximal end of the inner coil 170 within a length of the self-expandable member 172. In an embodiment, a distal portion of the inner catheter 114 extends partially inside the outer coil. In an embodiment, the release wire 152 is threaded through the half band 176 and is tucked underneath a portion of the self-expandable member 172 (e.g. some coils of the outer coil).

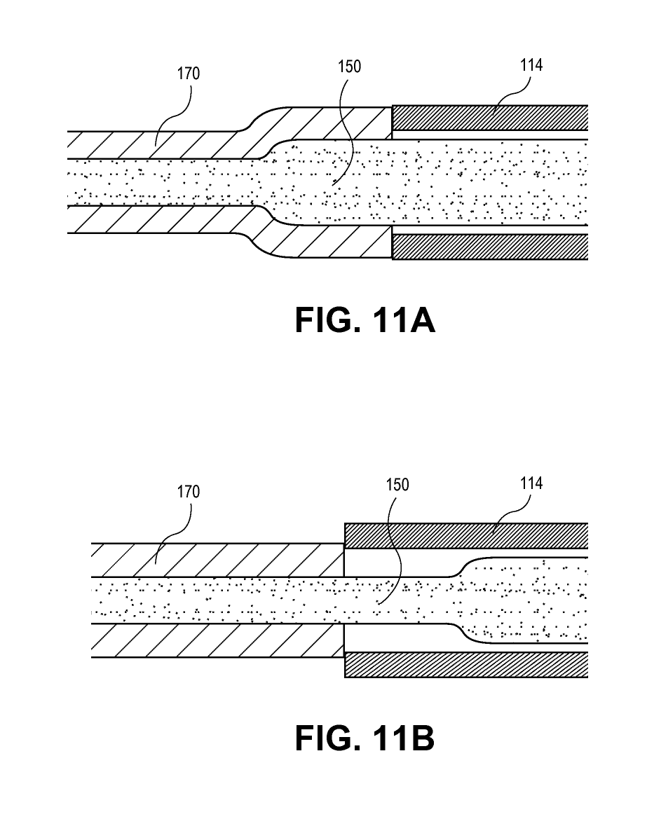

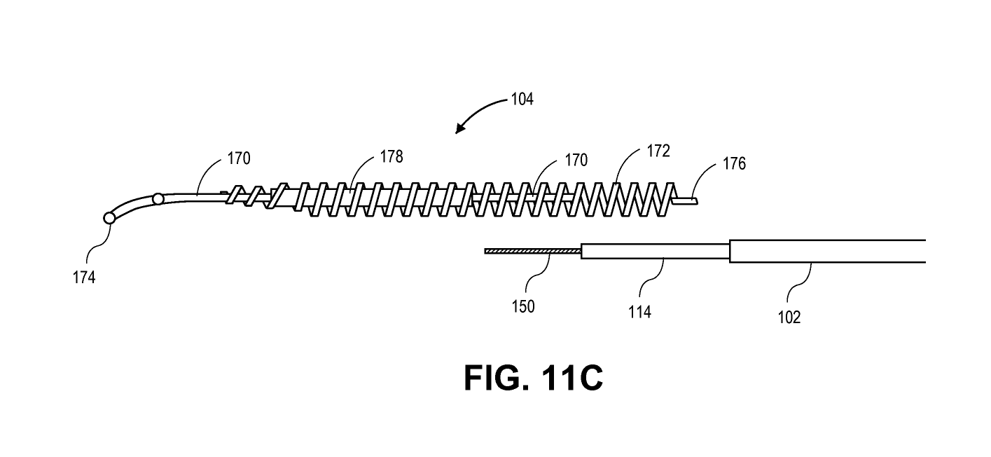

FIGS. 11A-11B are cross-sectional side view illustrations of withdrawing a friction fit core wire from an inner coil in accordance with an embodiment of the invention. The particular embodiment illustrated in FIG. 11A corresponds to the delivery system configuration prior to movement of the second release mechanism 110 (e.g. push button) from the first position to the second position, in accordance with an embodiment of the invention. The particular embodiment illustrated in FIG. 11B corresponds to the delivery system configuration after movement of the second release mechanism 110 (e.g. push button) from the first position to the second position, in accordance with an embodiment of the invention. As illustrated, prior to actuation of the second release mechanism 110, the core wire 150 is in a friction fit relationship with the inner coil 170. Upon actuation of the second release mechanism, the core wire 150 is withdrawn as the inner coil 170 abuts against the inner catheter 114 which keeps the occlusion device in place during withdrawal of the core wire 150.

FIG. 11C is a side view illustration comparing the relative dimensions of a disengaged and expanded occlusion device side-by-side with the distal portion of a delivery system after withdrawal of the release wire and core wire in accordance with an embodiment of the invention. The particular embodiment illustrated in FIG. 11C corresponds to movement of the second release mechanism 110 (e.g. push button) from the first position to the second position, in accordance with an embodiment of the invention. As shown, upon withdrawal of the release wire 152, the self-expandable member 172 (e.g. outer coil) self-expands, assuming the illustrated configuration of the occlusion device 104. It is to be appreciated, that while the occlusion device 104 and remainder of the delivery system are illustrated in a side-by-side manner that the core wire 150 and inner catheter 114 are partially within an inside of the occlusion device 104. In an embodiment, after actuation of the push button, the release wire is withdrawn past and within a distal end of the delivery catheter 102. The core wire 150 is likewise withdrawn an equivalent length. In the particular embodiment illustrated however, the core wire 150 is not required to be withdrawn completely from the occlusion device, and need only be withdrawn a distance sufficient to disengage from the occlusion device. For example, in an embodiment, the core wire 150 is engaged in a friction fit relationship with the inner coil 170 prior to actuating the push button. Upon actuation of the push button, sufficient spring force is generated by the extension spring to overcome the force retaining the release wire 152 in the wound-down configuration and the force retaining the core wire 150 in friction fit relationship with the inner coil. The inner catheter 114 functions as a backstop to retain the occlusion device 104 in place as the release wire 152 and the core wire 150 are withdrawn. Upon withdrawal of the release wire 152 and core wire 150 the delivery system including the core wire 150, inner catheter 115, release wire 152 and delivery catheter 102 can be freely withdrawn from the expanded occlusion device 104.

In accordance with embodiments of the invention, the various dampening configurations can be incorporated in a spring assembly to dampen a spring force from the extension spring in order to reduce or eliminate a resultant impact force transferred to the handle or the occlusion device. The impact force can potentially be derived from different sources such as an instantaneous release of energy as the extension spring jolts into action after being released, the extension spring reaching its free length, or the spring assembly coming to an abrupt stop.

As described above, upon actuation of the push button, sufficient spring force is generated by the extension spring to overcome the force retaining the release wire 152 in the wound-down configuration and the force retaining the core wire 150 in friction fit relationship with the inner coil. Specifically, the initial spring force of the extension spring in the extended state must be greater than the initial release force retaining the release wire and core wire. In an embodiment, the target initial spring force is at least approximately 2.5 times the target initial release force. In an embodiment, the target initial spring force is approximately 2.5 times to 3.5 times the target initial release force.

In one embodiment, an occlusion device is loaded in a delivery system 100 as described above. The core wire 150 is in a friction fit relationship with the inner coil of the occlusion device, and the release wire 152 is threaded through the half band 176 and tucked underneath the distal 0.06 inches of the outer coil. In such an embodiment, it has been determined that the initial release force that must be overcome to begin withdrawing both the release wire and core wire from the occlusion device is approximately 0.8 lbs.

In application, it is expected to have a variation in initial release force between delivery systems, and manufacturers. Likewise it is expected to have a variation in the initial spring force of the extension spring between extension springs, and manufacturers. In accordance with some embodiments, it has been determined that a target initial spring force of at least 2 lbs may achieve a sufficient confidence interval (CI) of at least a 95% confidence level that contraction of the extension spring will deploy the occlusion device. In an embodiment, the target initial spring force is at least approximately 2.5 times the target initial release force. In an embodiment, the target initial spring force is approximately 2.5 times to 3.5 times the target initial release force to achieve a sufficient CI of at least 95%.

It is to be appreciated, that while the target initial spring force may be 2.5 times to 3.5 times the target initial release force in such an embodiment, that the overall work of the contracting extension spring may be significantly greater than the overall work required to withdraw the release wire 152 and core wire 150 from the occlusion device. Accordingly, in accordance with embodiments of the invention greater than 95% of the overall work of the extension spring associated with contracting the extension spring may be dampened by one or more dampening mechanisms, while less than 5% of the overall work of the extension spring is countered by work associated with friction forces from withdrawal of the release wire and core wire from the occlusion device. This may be at least partially attributed to having a much longer spring contraction distance compared to the initial release force withdrawal distance. In an embodiment, the extension spring contraction distance is approximately 0.9 inches, compared to distance of 0.06 inches that the release wire 152 is tucked underneath the outer coil of the occlusion device. Such an approximation also assumes the friction fitting distance of the core wire and the inner coil of the occlusion device is less than 0.06 inches.

As described above, embodiments of the invention describe various dampening configurations that can be incorporated in a spring assembly to dampen a spring force of an extension spring to reduce or eliminate an impact force on the handle or occlusion device. In some embodiments, the dampening mechanisms are designed to dampen an initial impact force that could be derived from the instantaneous release of energy once with extension spring jolts into action after being released. In some embodiments, the dampening mechanisms are designed to gradually dampen the spring force in order to bring the extension spring to a gradual stop and dampen an impact force that could be derived from the extension spring reaching its free length, or the spring assembly coming to an abrupt stop. In some embodiments, the dampening mechanisms are designed to provide an increased drag force coefficient in order to bring the extension spring to a gradual stop and dampen the impact force.

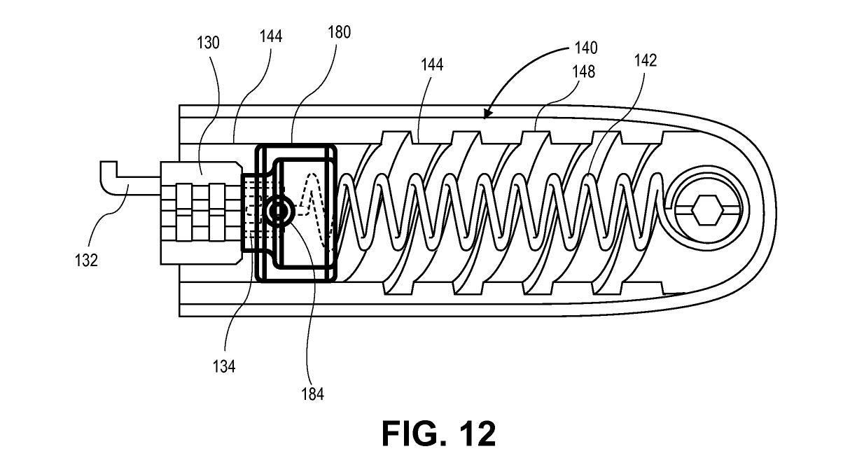

FIG. 12 is a schematic side view illustration of spring assembly 140 including an extension spring 142, damper ring 180, and helical track 148 in accordance with an embodiment of the invention. In operation, upon release of the latch 132 connected with the delivery wire holder 130, the extension spring 142 is allowed to contract pulling on the eyelet or hook 134 connected with the delivery wire holder 130 and drawing the delivery wire holder 130 into bore 144, similar to the movement of a piston. Movement of the delivery wire holder 130 in turn exerts a force on a damper ring 180 that travels along helical track 148 formed in a sidewall of the bore 144. In this aspect, implementation of a helical track 148 utilizes rotational motion to slow the linear motion of the extension spring. This enables a shorter linear length for the required amount of dampening, and utilizes available space within the handle.

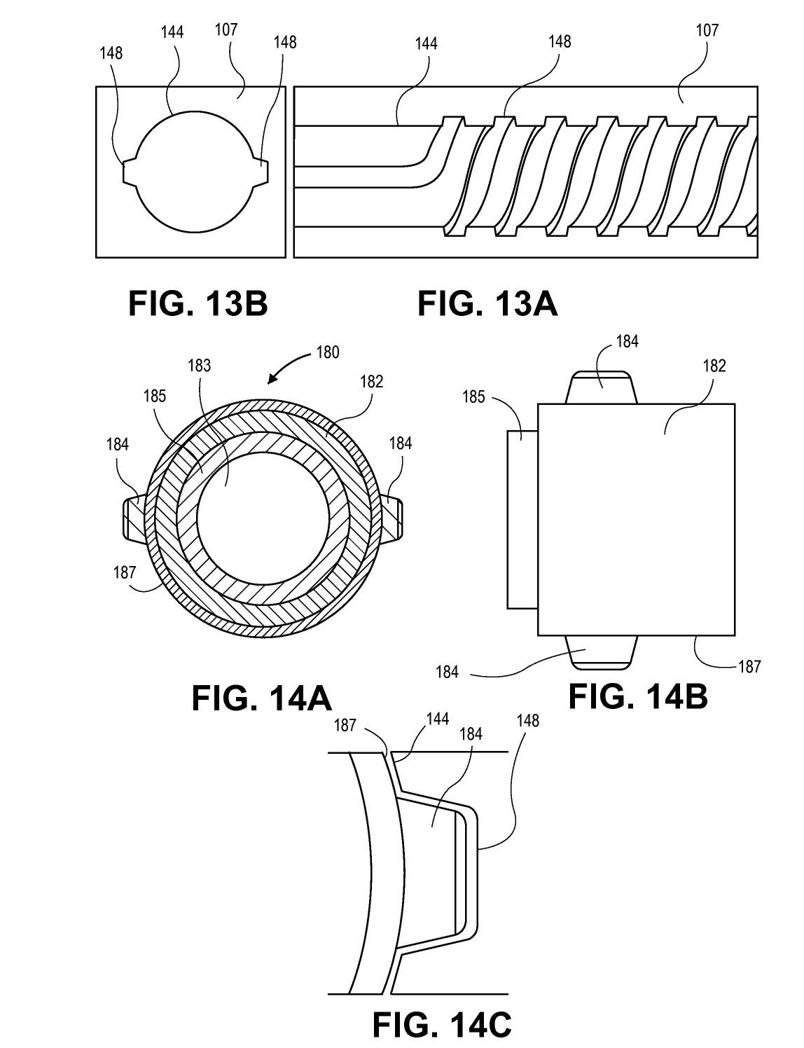

FIG. 13A is a side view illustration of a helical track formed in a sidewall of a bore with other features from FIG. 12 removed, in accordance with an embodiment of the invention. As illustrated, the track 148 may initially contain a straight portion, prior to assuming the helical arrangement in the proximal direction of the handle. FIG. 13B is a cross-sectional side view illustration of a helical track 148 formed in a sidewall of a bore 144 in accordance with an embodiment of the invention. In one embodiment, the bore 144 and helical track are formed from casing 107 or protruding features 112 in the handle.

FIG. 14A is a top view illustration of a damper ring 180 including one or more lugs 184 in accordance with an embodiment of the invention. FIG. 14B is a side view of the damper ring of FIG. 14A. In the particular embodiment illustrated, damper ring 180 includes a distal end surface 185 having an opening 183, and a tubular body 182. In an embodiment, the tubular body includes a circumferential side surface 187 and one or more lugs 184 extending from the circumferential side surface 187. Referring back to FIG. 12, in an embodiment, the opening 183 is slidable over the eyelet or hook 134 connected with the delivery wire holder 130, and the distal end surface 185 abuts against the delivery wire holder 130.

FIG. 14C is a close-up view of a lug within a helical track in accordance with an embodiment of the invention. As shown, the circumferential side surface 187 and one or more lugs 184 have a male-female relationship with the bore 144 and one or more helical tracks 148, respectively. In this manner, the damper ring 180 rotates along the one or more helical tracks as the damper ring 180 slides linearly within the bore. For example, if the damper ring 180 includes two lugs 184, the bore 144 has two corresponding helical tracks 148 in an embodiment such that the damper ring 180 travels along a double helical track.

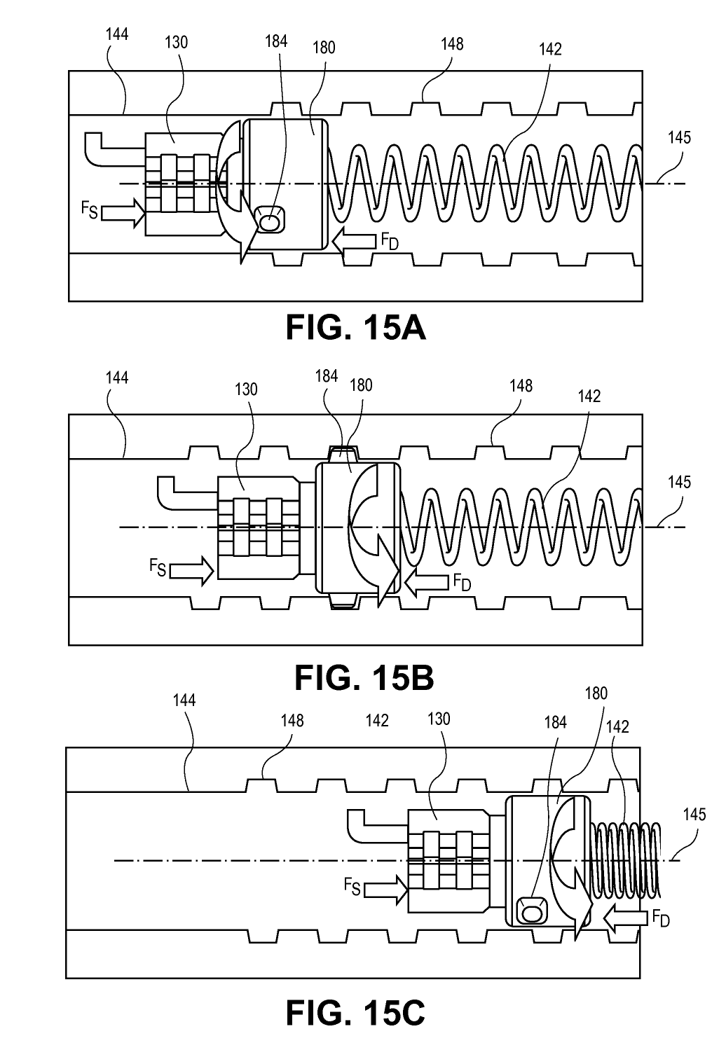

FIGS. 15A-15C are schematic side view illustrations of a delivery wire holder 130 and damper ring 180 traveling along an axial length of a bore 144 during linear contraction of an extension spring 142 while the damper ring rotates about a double helical track in the bore in accordance with an embodiment of the invention. Briefly referring back to FIG. 12, prior to actuation of the push 110, the one or more lugs 184 are located within the straight portion of the helical track 184. FIGS. 15A-15C illustrate the gradual motion of the spring assembly immediately after actuation of the push 110. As illustrated in FIG. 15A after traveling a short distance in the straight portion of the helical track 184, the one or more lugs 184 are forced into the helical portion of the helical track 184 by the delivery wire holder 130. In another embodiment, the one or more lugs 184 are already within a helical portion of the helical track 184 prior to actuation of the push 110. Referring to all of FIGS. 15A-5C, since the one or more lugs 184 are engaged with the one or more helical tracks 148, the damper ring 180 is forced to rotate about the axis 145 of the bore as it is pushed down the cylindrical bore 144. The geometry of the damper ring allows it to rotate in the cylindrical bore while the geometry of the bore constrains and controls the damper ring's rotation and allows for the damper ring to travel linearly down the bore as it rotates. This rotation motion of the damper ring 180 results in a drag force (F_{D}) in the opposite direction of the spring force (F_{S}), which effectively dampens the energy provided by the extension spring 142. As the extension spring continues to contract and gets shorter, its energy dissipates (spring forces decrease linearly) and is gradually dampened, thus gradually slowing down the speed of the spring assembly and decreasing the impact energy.

A variety of additional configurations can be implemented in combination with, or alternative to, the helical track to gradually slow down motion of the contracting extension spring, thereby reducing the impact force in accordance with embodiments of the invention. In some embodiments, an increased drag force coefficient is provided in the spring assembly.

FIGS. 16A-16B are side view illustrations of a helical track with differing pitch angles in accordance with embodiments of the invention. The pitch of the helical track can be changed to increase or decrease drag force coefficient along the helical track. A looser pitch helix has a lower pitch angle relative to the axis 145 than a tighter pitch helix. The higher the pitch angle is, the higher the drag force (F_{D}) will be, and the higher the drag force coefficient also. A pitch angle can be chosen from a range between 1 degree to 89 degrees to customize the mechanism to dampen a range of spring forces and energies. In an embodiment, the pitch angle of the helical track is varied from a low pitch to a higher pitch from a distal to proximal location in the bore. In such a configuration the pitch of the helical track is tightened along an axial length of the bore to achieve a more gradual ramp up of a drag force coefficient to slow down the spring assembly more gradually.

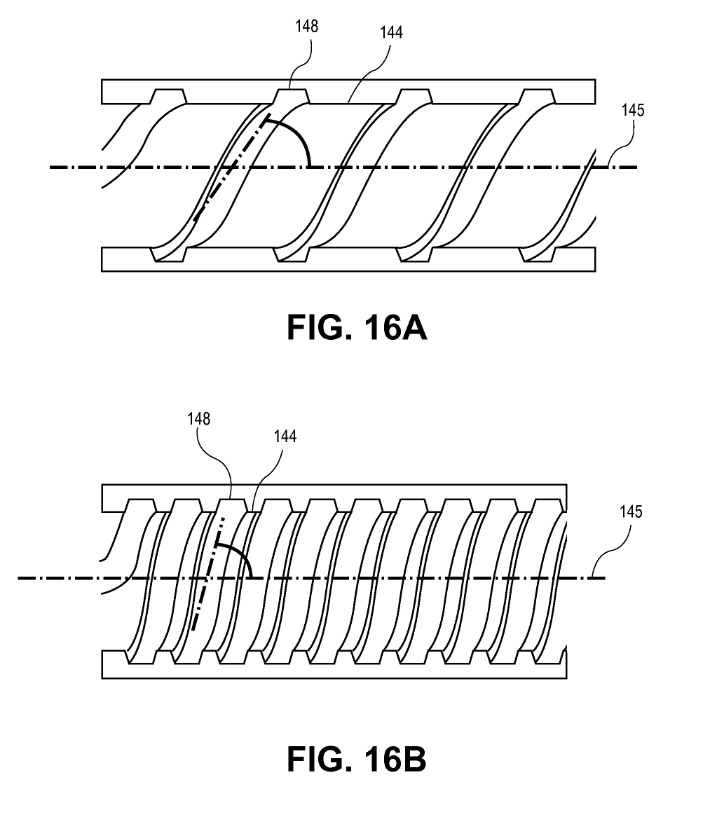

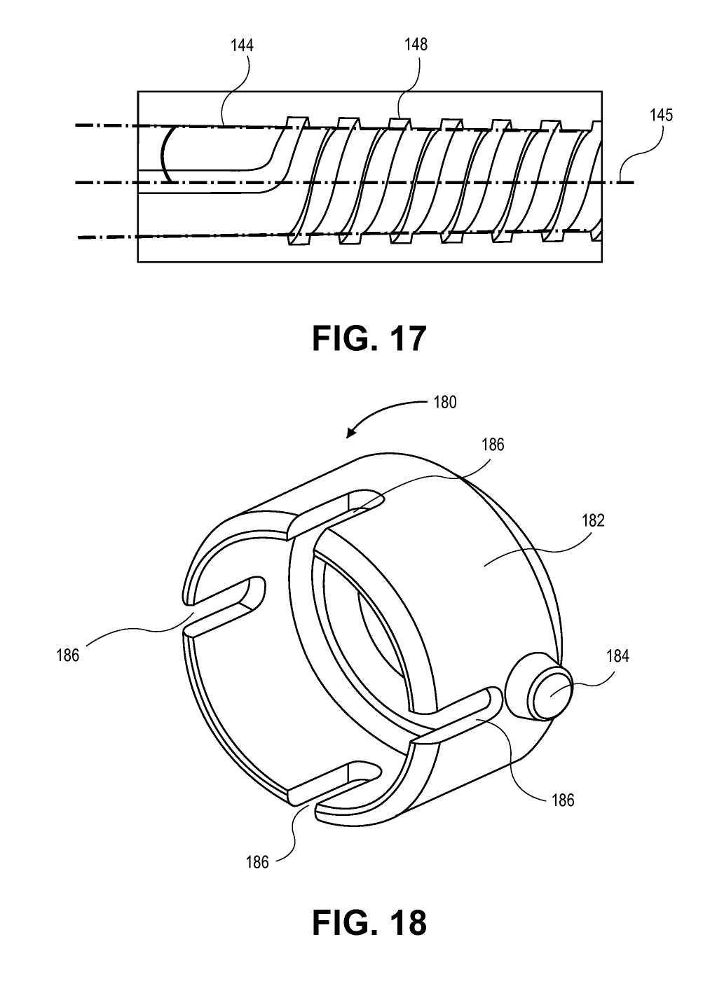

FIG. 17 is a side view illustration of a reduced diameter along an axial length of a bore in accordance with an embodiment of the invention. As illustrated in FIG. 17, the bore 144 is made to have a slight taper to it. As the inside diameter of the tapered bore decreases, there is less room for the damper ring 180 to move into. Eventually, the damper ring runs out of room and is squeezed into a smaller space than it can fit into, and is forced to come to a stop. This effectively increases the dampening energy of the spring assembly compared to just the cylindrical bore and helical tracks alone. The tapered bore guarantees the spring assembly will dampen all of the energy release from the extension spring and brings the spring assembly to a more controlled stop. In an embodiment, the taper angle can even be set such that the linear distance travelled can be pre-determined. The larger the taper angle, the shorter the linear distance travelled will be. In such a configuration that taper angle of the tapered bore achieves a more gradual ramp up of a drag force coefficient to slow down the spring assembly more gradually and bring the spring assembly to a controlled stop.

FIG. 18 is an isometric view illustration of a damper ring including slots around a circumference of a tubular body of the damper ring. As illustrated, one or more slots 186 are formed in a circumferential side surface 187 of tubular body 182 of the damper ring 180. When used in conjunction with a tapered bore, a slotted damper ring can increase dampening of the spring assembly. The slots 186 can be arranged around the circumference of the tubular body 182 and spaced evenly. The slots allow the tubular body 182 of the damper ring to flex, so it does not come to a sudden stop in the tapered bore 144. Instead, the tubular body 182 flexes as it is squeezed by the taper and comes to a stop in a smoother, less jolting manner.

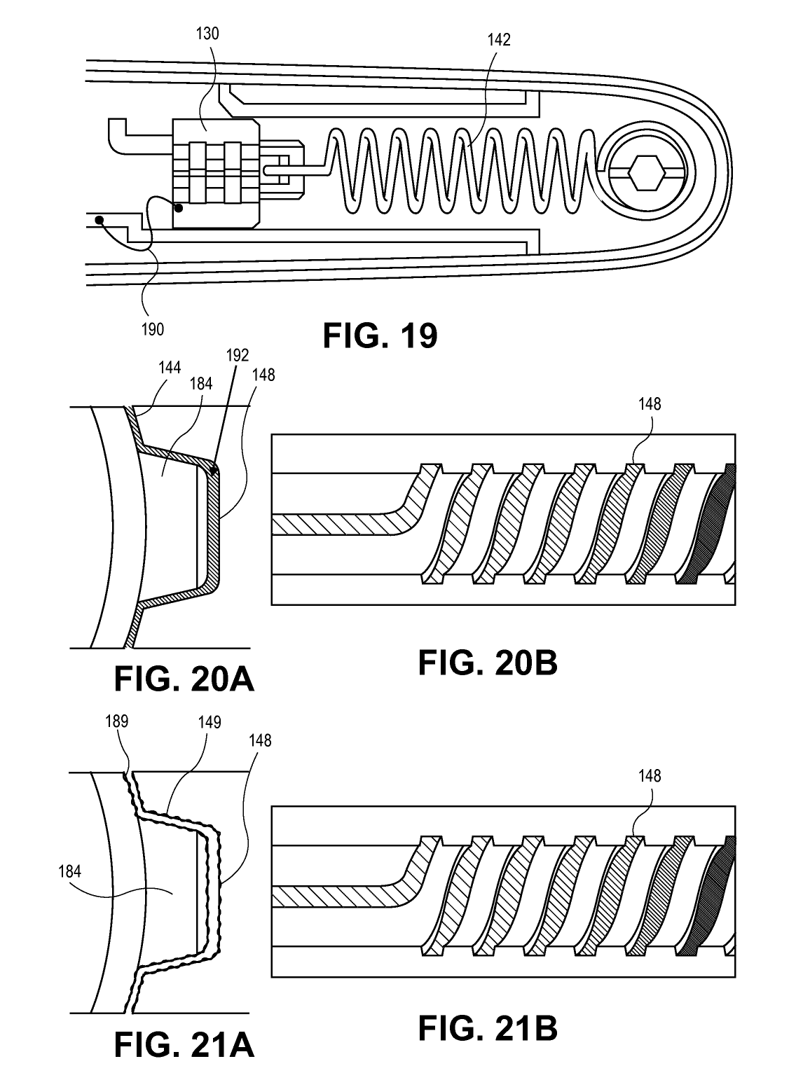

FIG. 19 is a side view illustration of a spring assembly including an elastic band 190 that couples a delivery wire holder 130 to a handle in accordance with an embodiment of the invention. In such an embodiment, the elastic band 190 provides resistance force as the extension spring contracts and pulls the delivery wire holder 130 from its starting point. In such a configuration the elastic band may assist in providing a more gradual ramp up of a drag force coefficient to slow down the spring assembly more gradually.

FIG. 20A is a close-up view of a dampening grease or fluid within a helical track in accordance with an embodiment of the invention. As illustrated, the dampening grease or fluid 192 can be located between the mating surfaces of the dampening ring and the cylindrical bore 144, as well as the one or more lugs 184 and the one or more helical tracks 148. FIG. 20B is a side view illustration of a dampening grease or fluid within a helical track in which a viscosity of the dampening grease 184 increases along an axial length of the bore in accordance with an embodiment of the invention. In the particular embodiment illustrated, the increase in viscosity of the dampening grease or fluid 184 is illustrated as the helical track 148 is darkened along an axial length of the bore 144. In such a configuration the dampening grease or fluid may assist in providing a more gradual ramp up of a drag force coefficient to slow down the spring assembly more gradually.

FIG. 21A is a close-up view of textured surfaces of a cylindrical bore, helical track, and mating surfaces of a damper ring in accordance with an embodiment of the invention. In the embodiment illustrated, the cylindrical bore and helical track can have a textured surface 149, and the mating surfaces of the dampening ring may also have a textured surface 189. However it is not required for both mating surfaces to be textured, and only of the mating surface can be textured in another embodiment. Likewise, only one of the bore or lugs or corresponding mating surface is textured in one embodiment, though both may be textured. Texturing of one or both of the mating surfaces in such as way may increase the friction between the mating surfaces as the damper ring is pushed through the bore. In an embodiment, the drag force coefficient increases with the amount of texturing. A suitable texture can be selected to achieve the amount of dampening energy output. In an embodiment, the texture can be varied from smoother to rougher. FIG. 21B is a side view illustration of increased texture roughness along an axial length of the bore in accordance with an embodiment of the invention. In the particular embodiment illustrated, the increase in roughness of the textured surface(s) is illustrated as the helical track 148 is darkened along an axial length of the bore 144. In such a configuration the textured surfaces may assist in providing a more gradual ramp up of a drag force coefficient to slow down the spring assembly more gradually.

Until this point the spring assembly has been described and illustrated as being an integral component of the delivery system 100. In the embodiments illustrated in FIGS. 22A-22B, a spring assembly 240 is illustrated as a self-contained modular unit. FIG. 22A is a perspective view of a self-contained modular spring assembly 240 in accordance with an embodiment of the invention. In such an embodiment, the spring assembly 240 can be a unit that is useful for a variety of applications including a delivery system such as delivery system 100, as well as other applications that require linear motion, much like a gas spring or an air cylinder. FIG. 22B is a schematic side view illustration of a self-contained modular spring assembly 240 in accordance with an embodiment of the invention. As illustrated, the bore 244, helical track 248, extension spring 242, and damper ring 280 are all contained within the housing 207. Rather than a delivery wire holder 130, the spring assembly 240 includes a piston 230 that similarly travels within bore 244, and a coupler 201 such as an eyelet or hook for coupling the spring assembly 240 to another component, such as a delivery wire holder 130 of a modified delivery system 100.

In an embodiment, the self-contained modular spring assembly 240 can be secured within the casing 107 of a handle 106 described above. In such an embodiment, the self-contained modular spring assembly 240 may be operably coupled with a delivery wire holder 130 within the handle 106 with coupler 201. The self-contained modular spring assembly 240 may be activated by a mechanism similar to that described above, where the second release mechanism 110 (e.g. push button) locks the delivery wire holder 130 into a static first position. The delivery wire holder 130 can be coupled to the self-contained modular spring assembly 240 via coupler 201, which comes pre-charged. For example, extension spring 242 could be held in the extended configuration as illustrated in FIG. 22B using any suitable locking mechanism 290. After loading into the self-contained modular spring assembly 240 the handle of delivery system 100 and coupling the coupler 201 to the delivery wire holder 130, the locking mechanism can be released so that the second release mechanism 110 (e.g. push button) locks the delivery wire holder 130 and coupler 201 into a static first position. Upon actuation of the second release mechanism 110, coupler 201 and delivery wire holder 130 is drawn into the housing 207 by linear contraction of the extension spring 242.

In the foregoing specification, various embodiments of the invention have been described. It will, however, be evident that various modifications and changes may be made thereto without departing from the broader spirit and scope of the invention as set forth in the appended claims. The specification and drawings are, accordingly, to be regarded in an illustrative sense rather than a restrictive sense. Hence, the scope of the present invention is limited solely by the following claims.

What is claimed is:

1. A delivery system comprising:
a delivery catheter;
an occlusion device;
a handle coupled with the delivery catheter, the handle comprising a spring-loaded release mechanism movable from a first position to a second position to cause withdrawal of a release wire from the occlusion device.

2. The delivery system of claim 1, wherein the occlusion device comprises a self-expandable member.

3. The delivery system of claim 2, further comprising a half band coupled with the self-expandable member.

4. The delivery system of claim 3, wherein the release wire is threaded through the half band and holds the self-expandable member in a wound-down static state prior to movement of the spring-loaded release mechanism from the first position.

5. The delivery system of claim 4, wherein movement of the spring-loaded release mechanism from the first position to the second position withdraws the release wire from the half band causing the self-expandable member to expand.

6. The delivery system of claim 4, wherein movement of the spring-loaded release mechanism from the first position to the second position withdraws a distal end of the release wire into a distal end of the delivery catheter.

7. The delivery system of claim 1, further comprising a first release mechanism, and movement of the first release mechanism from a first position to a second position withdraws a rack that is coupled with the delivery catheter to withdraw the delivery catheter from over the occlusion device.

8. The delivery system of claim 1, wherein the release wire is coupled with a delivery wire holder that is coupled to a spring assembly.

9. The delivery system of claim 8, wherein the spring-loaded release mechanism is a push button, and movement of the push button from the first position to the second position allows the spring assembly to withdraw the delivery wire holder and cause withdrawal of the release wire from the occlusion device.

10. The delivery system of claim 8, wherein the spring assembly comprises a damper ring and an extension spring, and a spring force associated with linear contraction of the extension spring is dampened by a drag force associated with rotational motion of the damper ring along a helical track in a bore.

11. The delivery system of claim 10, wherein the bore and helical track are formed from a casing of the handle.

12. The delivery system of claim 10, wherein the damper ring comprises one or more lugs that travel within a matching quantity of paths in the helical track.

13. The delivery system of claim 10, wherein a drag force coefficient increases as the damper ring travels axially within the bore.

14. The delivery system of claim 10, wherein a pitch of the helical track is tightened along an axial length of the bore.

15. The delivery system of claim 10, wherein an inside diameter of the bore is reduced along an axial length of the bore.

16. The delivery system of claim 15, wherein the damper ring includes slots around a circumference of a tubular body of the damper ring.

17. The delivery system of claim 10, wherein a pitch of the helical track is tightened along an axial length of the bore, an inside diameter of the bore is reduced along the axial length of the bore, and the damper ring includes slots around a circumference of a tubular body of the damper ring.

18. The delivery system of claim 10, wherein a surface roughness of the helical track increases along an axial length of the bore.

19. The delivery system of claim 10, further comprising a dampening grease within the helical track, wherein a viscosity of the dampening grease increases along an axial length of the bore.

20. The delivery system of claim 10, further comprising an elastic band that couples the delivery wire holder to the handle to provide an increase in a drag force coefficient as the extension spring linearly contracts.

21. The delivery system of claim 9, wherein the release wire and a core wire are coupled with the delivery wire holder.

22. The delivery system of claim 21, wherein movement of the push button from the first position to the second position allows the spring assembly to withdraw the delivery wire holder and cause simultaneous withdrawal of the release wire and the core wire from the occlusion device.

23. A spring assembly comprising:
a bore and a helical track along a wall of the bore;
a piston;
a damper ring connected with the piston; and
an extension spring coupled with the piston;
wherein linear contraction of the extension spring causes axial motion of the piston and the damper ring within the bore and rotational motion of the damper ring along the helical track.

24. The spring assembly of claim 23, wherein the damper ring includes a body and one or more lugs that travel within a matching quantity of paths in the helical track.

25. The spring assembly of claim 23, wherein a pitch of the helical track is tightened along an axial length of the bore.

26. The spring assembly of claim 23, wherein an inside diameter of the bore is reduced along an axial length of the bore.

27. The spring assembly of claim 23, wherein the damper ring includes slots around a circumference of a tubular body of the damper ring.

28. The spring assembly of claim 23, wherein a pitch of the helical track is tightened along an axial length of the bore, an inside diameter of the bore is reduced along the axial length of the bore, and the damper ring includes slots around a circumference of a tubular body of the damper ring.

29. The spring assembly of claim 23, wherein a surface roughness of the helical track increases along an axial length of the bore.

30. The spring assembly of claim 23, further comprising a dampening grease within the helical track, wherein a viscosity of the dampening grease increases along an axial length of the bore.

31. The spring assembly of claim 23, further comprising an elastic band that couples the piston to a housing or casing to provide an increase in a drag force coefficient as the extension spring linearly contracts.

32. The spring assembly of claim 23, wherein the piston is a delivery wire holder.

33. The spring assembly of claim 23, wherein the spring assembly is a self-contained modular spring assembly, and further comprising a coupler coupled with piston and extending outside of a housing.

34. The spring assembly of claim 33, wherein the coupler comprises an eyelet or hook.
